(19) United States
(12) Patent Application Publication
(10) Pub. No.: US 2002/0182748 A1
(43) Pub. Date: Dec. 5, 2002

(54) Method and device for testing for Bence-Jones Protein

(76) Inventor: Paul C. Reardon, Surrey (GB)

Correspondence Address: ST. ONGE STEWARD JOHNSTON & REENS, LLC, 986 BEDFORD STREET, STAMFORD, CT, 06905-5619, US

(21) Appl. No.: 09/823,183

(22) Filed: Mar. 30, 2001

(51) Int. Cl. G01N 33/558

(52) U.S. Cl. 436/514

(57) ABSTRACT

A method for the determination of the presence of free light chains (Bence Jones protein) in a unconcentrated and undiluted urine sample is provided in which the sample is reacted with an anti-free light chain antiserum reagent, where the presence of the free light chains is revealed by improved specific binding assay methods, including kits and devices utilizing chromatographically mobile specific binding reagents labeled with colloidal particles. Specific binding reagents labeled with colloidal particles such as gold may be subjected to rapid chromatographic solvent transport on chromatographic media by means of selected solvents and chromatographic transport facilitating agents.

### FIELD OF THE INVENTION

The present invention relates generally to assay devices and specifically to those devices making use of chromatographic techniques in conducting specific binding assays for the determination of the presence of light chains (free & bound) in untreated human urine. Test kits for kappa, lambda and combination kits are demonstrated.

### BACKGROUND OF THE INVENTION

Tests for determining the presence of light chains (free and bound) are presently known in the art, as is the use of chromatographic strips for the detection of protein. The use of such chromatographic strips for the detection of light chains however has not been heretofore proposed or used for the detection of light chains in untreated urine. This invention solves the problems of insensitivity, unreliability, high cost, and time consumption of the typical state of the art tests, as presently known.

The present invention provides an improved, less expensive, easy to use test for the detection of free and bound light chains, as well as simplified test procedures without the prior problems of known tests which can be laborious, time consuming, costly, and require skill in interpretation. The chromatographic method and devices of the present invention is capable of reliably sensing free and bound light chains with increased sensitivity and require minimal interpretation. The improved method and devices of the present invention enable physicians and assistants thereof the ability to routinely perform such a test for the detection of free light chains in a doctor's office without the wasted time and expense of sending urine samples to a lab for laborious examination. In cases where the labor-intensive state of the art tests have been performed, the claimed invention can also be used to quickly verify any results.

It is known that an immunoglobulin is schematically made up of two heavy chains and two light chains. Determinations of the presence of the free light chains, also called Bence Jones proteins, which pass into the urine is of great interest from a diagnostic viewpoint.

Gammopathies characterized by the proliferation of B cell clones, yield increased clonal production of immunochemicals. In addition to the primary effects of the gammopathy, secondary consequences dependent on the class of clonal immunochemical produced may emerge. The preferential production of single immunoglobulin clones will result in immunodefiency as a result of normal immunoglobulin production, and the production of toxic free light chains will cause renal disease even at low-level depositation. Immunological pathologies caused by the presence of free light chains in the urine, which is the consequence of the increase thereof in the blood, is associated with and can be summarized as (a) the presence of monoclonal free light chains, i.e. immunoproliferative illnesses such as multiple myeloma, micromolecular myeloma, Waldenstrom's macroglobulinemia, chronic lymphatic leukemia and primitive amyloidosis; and (b) the presence of polyclonal free light chains, i.e. hyperimmune illnesses such as systemic lupus erythematosus, acute rheumatoid arthritis and secondary amyloidosis.

The presence of free light chains in urine presupposes their anomalous increase in the serum of the subject but, given their low molecular weight, free light chains pass through the glomerular filter and do not persist in the blood. It is therefore necessary to perform an indirect investigation, ascertaining their presence in the urine.

Known diagnostic methods based on ascertainment of free light chains in the urine are of great interest but at present are blocked by the difficulties of performance of such an investigation. Current techniques such as that described in U.S. Pat. No. 5,141,877 to Massaro, herein incorporated by reference, necessarily require concentration of the sample because of the relatively small percentage of free light chains in the organic liquid even with serious pathological conditions of the subject. Electrophoretic examination of the unconcentrated sample results in unacceptably low sensitivity and the resulting unreliability. The time necessary for concentration is added however to the time required for electrophoretic analysis with the obvious drawbacks. The analysis performed on the concentrated samples undoubtedly raises the reliability of the results without however achieving reasonable certainty. On the samples which prove suspect under electrophoresis it is therefore very advisable to perform immunofixation or immunoelectrophoretic tests, the laboriousness and cost, and requirement for interpretation of which are known, to achieve truly satisfactory levels of sensitivity and hence reliability of the analysis results.

Of interest to the present application is the disclosure of U.S. Pat. No. 4,727,037, issued to Ring, relating to a method for determining the class and subclass of a test antibody which demonstrates adsorbing on nitrocellulose paper or diazo paper in a predetermining spatial arrangement, antisera directed to isotype class and subclasses of an immunoglobulin; contacting said adsorbed isotype specific antisera with a fluid containing a test antibody to form a complex between the specific immunoglobulin heavy and light chains of the adsorbed antisera and said test antibody present in the fluid; and reacting the complex so formed with a chromogenic substrate to thereby identify the specific class or subclass of said test antibody.

U.S. Pat. No. 5,141,877 describes a method for the determination of the presence of free light chains (Bence Jones protein) in a urine sample in which the sample is reacted with an anti-free light chain antiserum reagent, where the presence of the free light chains is revealed by increase in turbidity of the reacted sample. By comparison with the turbidity of calibrators having predetermined concentrations reacted with anti-free light chain antiserum, a quantitative analysis of the amount of free light chains in the urine sample can be determined. The test samples are derived from patients having secretory micromolecular myeloma.

U.S. Pat. No. 5,569,608 describes a method for determining the concentration of analyte in a test fluid by immunochromatography techniques which involves quantitatively determining the signals from captured analyte/labeled binding partner complex by an instrument, e.g. a reflectance spectrometer. A reflectance reading is determined for the captured complex and uncomplexed labeled binding partner which is captured in a separate zone of the immunochromatographic strip and the ratio of these reflectances is used to provide additional quantification to the assay method.

U.S. Pat. No. 5,780,308 teaches a multizone test device for semiquantitatively determining the presence of at least a predetermined minimum concentration of an analyte in a test sample. The device utilizes a strip of porous material, said strip comprising a reagent zone and a capture site. The reagent zone is upstream from the capture site. The reagent further contains a soluble conjugate comprising a labeled analyte-specific binding member which binds the analyte to form a labeled analyte complex. In addition the reagent zone has a capture reagent comprising an unlabeled specific binding member attached to the porous material. The capture reagent binds the labeled analyte complex to form an immobilized labeled analyte complex. Further the reagent zone includes a soluble calibration reagent comprising an unlabeled specific binding member which blocks the binding of the analyte to the capture reagent, thereby controlling the proportion of the analyte that binds to said capture reagent. As a consequence the analyte in the test sample must exceed a minimum concentration before the immobilized labeled complex is formed. The calibration reagent is contained in said reagent zone. The capture reagent is immobilized at the capture site where the immobilized labeled complex is separated from the test sample, and where the presence of label associated with said immobilized labeled complex is detected to determine the presence of at least a predetermined minimum concentration of an analyte in the test sample.

U.S. Pat. No. 5,989,921 relates to a test device for determining the presence of a ligand in a liquid sample, the device has a casing defining an inlet and at least one window for viewing through a wall of said casing, a test strip disposed within said casing comprising a sorbent material which defines a flow path for transporting the liquid sample there along from said inlet to a test site and a control site, and, disposed upstream of said test site and said control site. Further this is a conjugate comprising a specific binder for the ligand and a colored particulate material. The test site comprises an immobilized first binding protein which binds specifically to the ligand, if the ligand is present in the liquid sample. The control site comprises an immobilized binder which binds said conjugate. The inlet, test site, and the control site are in lateral flow fluid communication along a flow path, such that after a liquid sample suspected to contain the ligand is applied to said inlet, said conjugate moves along said flow path and binds to said immobilized binder of said control site to produce a color visible to the unaided eye through at least one window indicative of a valid test result. If the ligand is present in the liquid sample, a specific binding reaction product comprising the ligand and said conjugate binds to the immobilized first binding protein of the test site to produce a color visible to the unaided eye through at least one window indicative of the presence of the ligand in the sample.

As stated above, known techniques are technically difficult, slow, and expensive and with a sensitivity below that specified as clinically significant (typical gel electrophoresis sensitivity is 30-50 mg/L, it is recognized that 10 mg/L BJP is significant). Since the presence of Bence Jones protein in the urine may not be supported or suggested symptomatically, a large percentage of positive samples remain unidentified in the early stages. The present invention relates generally to assay devices and specifically to those devices making use of chromatographic techniques in conducting specific binding assays for the determination of the presence of free light chains in untreated urine. In particular, this technique provides kits for rapid determination between whole antibody and free light chain, and between classes of light chain. The present invention provides improved assays that require little technical expertise, are rapid, highly sensitive and may be used either as a screen, or as a confirmatory step prior to immunoelectrophoresis, or immunofixation electrophoresis utilizing untreated human urine.

### SUMMARY OF THE INVENTION

It is the object of the present invention to provide a method, device, and various kits for a specific binding assay for the detection of free light chains in untreated urine wherein chromogenic mobile specific binding partners with colloidal particles which are capable of chromatographic solvent transport are impregnated and dried onto the conjugate pads of the devices. The conjugate pads are readily disposed upon a chromatographic test strip comprising a matrix through which a urine sample can flow by capillarity. The test strip comprises at least two reaction sites comprising pre-impregnated reactants. The first reaction site contains an immobilized specific binding reagent capable of immobilizing the chromogenic mobile specific binding partner in relation to the presence of an analyte within the urine sample. A control reaction site is also provided having pre-impregnated reactants comprising a specific binding reagent capable of binding immunochemicals.

The ability to impregnate conjugate pads with the labeled specific binding partners, which may then be resolubilized with untreated urine, along with the use of a chromatographic test strip makes possible the practice of a variety of assay procedures which avoid the use of labeled reagent addition steps. Both sandwich-type and competition-type assays may be conducted using the kits and strips of the present invention.

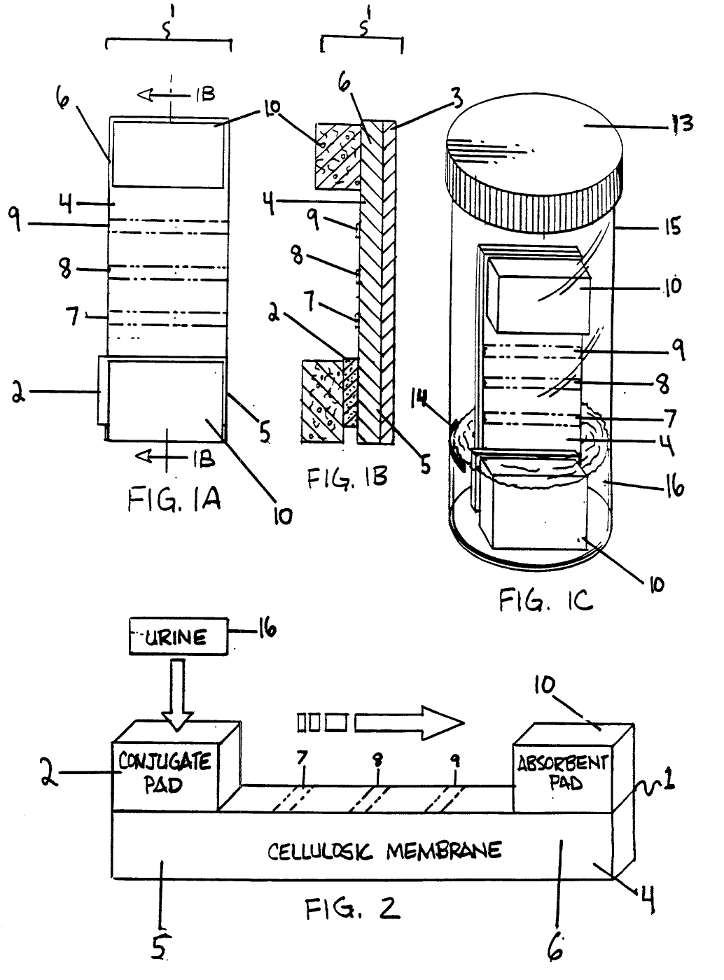

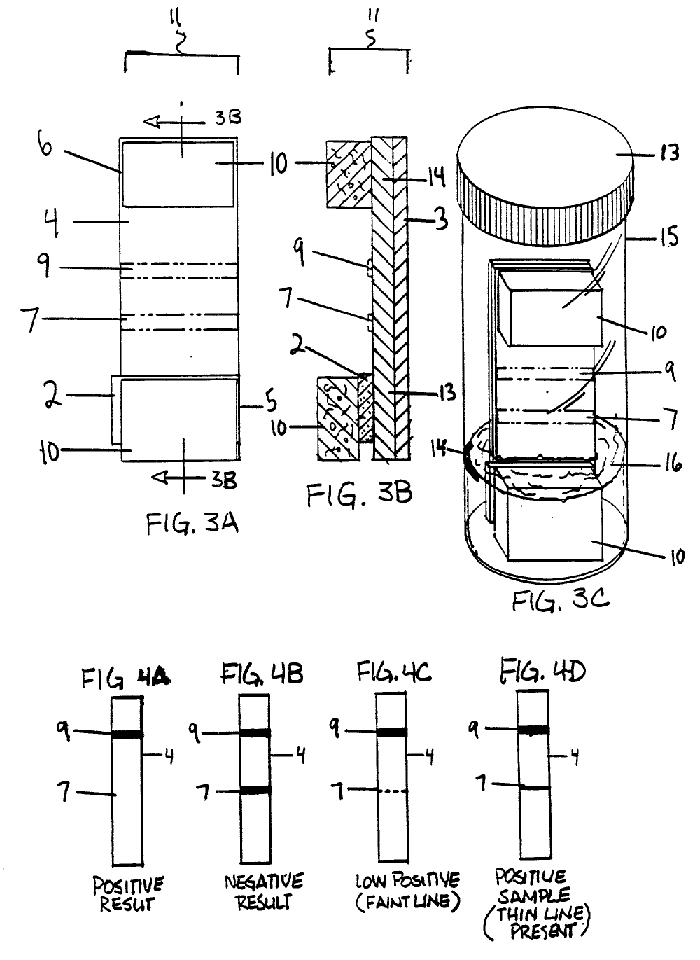

FIGS. 1a, and 3a are front plan views of two different forms of the test devices of the present invention.

FIGS. 1b, and 3b are cross-sectional views of the test devices shown in FIGS 1a, and 3a respectively.

FIGS. 1c and 3c are front plan views of a kit embodiment of the present invention showing the test device shown in FIGS. 1a, and 3a.

FIG. 2 is a schematic view of a sandwich type test device of the present invention.

FIG. 4 is a schematic view illustrating how to interpret results of the competitive assay of the present invention.

### DETAILED DESCRIPTION OF THE DRAWINGS

Sandwich Assay Devices

Referring to the drawings, FIGS. 1a, 1b, and 2 depict a joint assay test device (1) for the detection of free light chains (kappa and/or lambda), free and bound antibodies (kappa and/or lambda), and immunochemicals in urine sample (16) (treated and/or untreated) wherein a chromogenic mobile specific binding partner is impregnated and dried into a conjugate pad (2) by soaking the conjugate pad (2) in a solution containing the chromogenic mobile specific binding partner.

The chromogenic mobile specific binding partner is selected from anti-free and bound kappa and anti-free and bound lambda antibodies conjugated with a visible colloidal metal. Although other colloidal metals that one of skill in the art would use to bind to an antibody to make a visible color may work, gold is preferred with the present invention for it provides an easily discernable red band in relation to the presence of analyte. The chromogenic mobile specific binding partner may be one conjugated monoclonal antibody or a conjugated monoclonal antibody cocktail.

The device (1) may comprise a length of substrate material (3) upon which cellulosic membrane (4) is disposed. Cellulosic membrane (4) has a first end (5) at which chromatographic urine transport begins and a second end (6) at which chromatographic solvent transport ends. The length of cellulosic membrane (4) comprises a first reaction site (7), a second reaction site (8), and a control reaction site (9). Typically cellulosic membrane (4) is 60 mm. in length, and 6 mm. wide. All parameters on the cellulosic membrane (4) may be changed but the width will not affect the sensitivity (provided it is above ˜4 mm, where edge drag effects come in to play). Absorbent pad (10) is placed upon cellulosic membrane (4) opposite conjugate pad (2) such that capillary action can draw urine (16) from first end (5) to second end (6) thereby transporting urine (16) through reaction site (7), second reaction site (8), and control site (9). Absorbent pad (10) can be fabricated from any material that has a propensity to wick liquid such as a sponge or paper towel material and is typically 17 mm. long and 6 mm. wide. The size and thickness must be such that it provides enough capillarity to transport urine (16) from first end (5) to second end (6).

The first reaction site (7) is impregnated with a first immobilized specific binding reagent capable of reaction with and the immobilization of the chromogenic mobile specific binding partner when analyte such as free light chain is present in urine (16) sample. In a joint assay (2) the immobilized specific binding reagent is an anti-free light antibody capable of binding free kappa or lambda light chains in untreated urine samples harboring free light chains. The second reaction site (8) is downstream of the first reaction site (7) and is impregnated with a second immobilizing specific binding factor. Typically, the second immobilizing specific binding factor is anti-free and bound antibody capable of detecting whole antibodies in untreated urine samples harboring whole free and bound antibodies. The control reaction site (9) is further downstream than reaction site (8) and is impregnated with a third immobilizing specific binding factor. Typically a chemical capable of specifically binding to immunochemicals, such as Protein A is used for it is capable of effectively collecting and immobilizing antigen-antibody complexes by binding to the Fc section of immunoglobulins. Control reaction site (9) acts as a positive control and is relied upon to indicate that capillary action has carried test urine (16), chromogenic mobile specific binding partners, and analyte/chromogenic mobile specific binding partner complexes thereof throughout the length of the chromatographic test strip.

Referring now to FIG. 1c, a kit of the present invention is shown. Typically device (2) is obtained within reaction tube (15) having a cap (13). Reaction tube (15) has line (14) to ensure the correct aliquot of urine (16) is added to reaction tube (15). When device (2) is to be used, the device is removed from reaction tube (15). Urine (16) is added to line (14) to ensure the proper amount of urine (16) aliquot. Urine (16) may be untreated urine which means that it is urine collected directly from a test individual. Urine (16) is typically obtained by collecting a sample midstream from a urinating individual, and then adding an aliquot to reaction tube (15). Although the urine (16) aliquot may fall within the range of 100 microliters to 1 milliliter, 300 microliters is optimal for the instant invention. Upon the addition of urine aliquot (16) device (2) is placed back into reaction tube (15). Typically Cap (13) is reapplied to reaction tube (15) and the test is allowed to run. The cap (13) ensures a seal on reaction tube (15) providing for an improved disposal of biological waste, reducing the contact between the person using the kit and the biological chemicals and waste therein.

A method of use for device (1) of FIGS. 1a, 1b, and 2 comprises obtaining cellulosic membrane (4) and placing it laterally upon a surface. Conjugate pad (2) is placed upon first end (5), such that conjugate pad (2) is placed adjacent to first reaction site (7). Absorbent pad (10) is next placed on the opposite end of cellulosic membrane (4) upon second end (6) adjacent to control reaction site (9). A second absorbent pad (10) may be upon conjugate pad (2) to facilitate contacting test urine (16) with conjugate pad (2). The device (1) is next contacted with between 200 μl to 1 ml. and optimally 300 μl of untreated urine (16) at conjugated pad (2) such that untreated urine (16) will be transported or wicked through the length of cellulosic membrane (4) to absorbent pad (10) located at second end (6) by capillary action. When the untreated urine (16) passes into conjugate pad (2) the chromogenic mobile specific binding partner goes into solution and will react with antigen in an antigen positive urine sample. Antigen means free light chains.

When antigen is present a complex called the analyte/chromogenic mobile specific binding partner complex is formed. Typically antibody cocktails containing chromogenic monoclonal antibodies will be put in conjugate pad (2) for the detection of multiple analytes, forming multiple complexes. Such complexes are mobile and will migrate through cellulosic membrane (4) towards absorbent pad (10) due to the capillary action and flow of urine (16) throughout device (2).

The first reaction site (7) comprises an immobilized specific binding reagent impregnated onto the cellulosic membrane (4). Typical immobilized specific binding reagents include antiserum such as polyclonal anti-free and bound antibodies. In analyte positive samples antiserum binds to the complex thus immobilizing the complex resulting in a high concentration of chromogenic complex reagent in the first reaction site. When a high concentration of chromogenic complex reagent becomes immobilized in the first reaction site a visible band is formed.

The second reaction site is downstream from the first reaction site and is impregnated with antiserum capable of immobilizing chromogenic mobile specific binding partner complexed with whole antibody. When whole antibody antigen is present in urine (16), and a complex is subsequently formed in conjugate pad (2), reaction site (8) containing polyclonal anti-free and bound whole antibodies will immobilize said antibodies making them visible in high concentrations.

The third control reaction site (9) is located downstream of reaction site (8) and comprises a specific binding reagent capable of immobilizing the chromogenic mobile specific binding partner upon contact. Control reaction site (9) acts as an indicator that the test has worked and that at least urine (16) and mobile specific binding partner have been transported through the chromatographic strip. Control reaction site (9) will also immobilize complex that did not bind to first reaction site (7) and/or second reaction site (8). A high concentration of either bonded complex, or non-complexed mobile specific binding partner at control reaction site (9) will form a visible band indicating that the test has worked correctly.

Device (2) is preferably 60 mm. in length and 6 mm. wide. First reaction site (7) containing a first specific immobilizing reagent impregnated into membrane (4) is approximately 1 mm. in width, and 6 mm. in length as it extends across membrane (4). Second reaction site (8) and control reaction site (9) are striped adjacent to the first reaction site (7) approximately and respectively 5 mm. downstream from first reaction site (7). Conjugate pad (2) is typically made of glass paper and is approximately 5 mm. long and has a width of 6 mm. extending across membrane (4). Absorbent pad (10) is approximately 20 mm. long and is also 6 mm. wide extending across the width of membrane (4).

A variety of sandwich-type assay devices including dried labeled reagents and preferably including colloidal particle labeled reagents may be produced according to the invention. It is frequently desirable to avoid premature contact of analyte and sample materials with the reagents and contact of the reagents with each other. Thus, the relative mobility of the sample components and the various reagents or the site relationship between the zones may be selected such that the reagents and sample components mix at only the times and locations desired. U.S. Pat. No. 4,960,691 herein incorporated by reference discloses various methods and devices for conducting chromatographic solvent transport assays where it is desired to avoid contact of a labeled first reagent material (such as an anti-human immunoglobulin antibody) with sample material (such as serum) prior to the time at which the analyte antibody is immobilized against solvent transport at a reaction zone. Other non-analyte antibodies contained in the serum sample are cleared from the third zone by chromatographic solvent transport.

The assay devices of the present invention for the detection of free light chains, and immunochemicals in an untreated urine sample wherein a chromogenic mobile specific binding partner is impregnated and dried into conjugate pad are also suitable for the practice of competitive binding type assays. According to such methods, the immobilized second reagent is selected, as in sandwich-type assays, so as to specifically bind with the analyte of interest. The labeled mobile specific binding partner, however, is selected to be a specific binding analogue of the analyte that will bind competitively with the immobilized specific binding reagent. In carrying out competition type assays according to the invention, it is generally not necessary that the analyte and the colloidal particle labeled reagent be prevented from contacting each other prior to their contacting the immobilized specific binding reagent. Thus, the device may be designed so as to mix the analyte containing untreated urine sample and the chromogenic mobile specific binding partner.

Referring to the drawing, FIGS. 3a and 3b depict a test device (11) for conducting competitive binding assays for the detection of the analyte of interest such as free light chains in an untreated urine (16) wherein a chromogenic mobile specific binding partner is impregnated and dried into conjugate pad (2). Typically the chromogenic mobile specific binding partner in the competitive assay is selected from antibodies to the free and bound light chain classes that have been conjugated with colloidal gold. Device (11) comprises a length of chromatographic substrate material (12) with a first end (5) at which chromatographic solvent transport begins and a second end (6) at which chromatographic solvent transport ends. The length of material (12) comprises a first reaction site (7) and a control reaction site (9). First reaction site (7) is impregnated with immobilized specific binding reagent, which is typically a monoclonal light chain selected from free kappa, free and bound kappa, free lambda, and free and bound lambda. By selecting and utilizing a different immobilized specific binding reagent it is possible to create four assays in an array, each having a different first reaction site (7). The control reaction site (9) is downstream of the first reaction site (7) and is impregnated with a second reagent, which is capable of a selective binding reaction with both the analyte and the chromogenic mobile specific binding partner and complexes thereof so as to render the analyte and chromogenic mobile specific binding partner in immobilized form. Protein A is one such suitable reagent capable of binding immunochemicals and complexes thereof. The device further comprises an inert substrate (3) to which the length of chromatographic substrate material (12) is affixed. Typically this inert substrate is simply a backing material to a nitrocellulose membrane or cellulosic membrane.

According to a procedure for use of device (11) of FIGS. 3a, 3b, and 3c, cap (13) is removed from reaction tube (15) the device (11) is removed, an untreated urine (16) sample aliquot to be tested in an amount ranging between 0.200 ml. to 1.0 ml, optimally 0.300 ml. is added to reaction tube (15). Device (11) is again deposited within the reaction tube such that first end (5) and conjugate pad (2) is deposited directly into the untreated urine (16). The cap (13) is replaced. Upon contact of device (11) at its first end (5) into a reaction tube (15) of urine aliquot (16), the mobile specific binding partners go into solution and begin to flow with the urine (16). If the analyte of interest is present in urine (16), a reaction occurs where the analyte of interest and the chromogenic mobile specific binding partner will form a complex. The urine aliquot (16) then progresses through the length of the chromatographic substrate material (12) transporting the chromogenic mobile specific binding partner impregnated at the conjugate pad (2) and/or any complexes thereof to the first reaction site (7). There the analyte and chromogenic mobile specific binding partner compete to bind with the immobilized specific binding reagent for which they are both specifically reactive. If no analyte is present the chromogenic mobile specific binding partner will bind to the immobilized specific binding reagent and a visible line will form. Non-analyte components as well as unbound analyte and chromogenic mobile specific binding partner are transported away from the first reaction site (7) by means of the urine transport which continues until the chromatographic solvent is exhausted or the urine front reaches the second end (6) of the material. At the conclusion of the chromatographic solvent transport, the first reaction site (7) may be observed to determine the presence of chromogenic mobile specific binding partner immobilized at that location. The presence of chromogenic mobile specific binding partner at that location may then be related to the presence of analyte in the sample. Where the chromogenic mobile specific binding partner is labeled with colloidal particles, its presence at the first reaction site, as well as the presence of analyte/chromogenic mobile specific binding partner complex may be observed directly.

The presence of chromogenic mobile specific binding partner and analyte complexes thereof at control reaction site (9) is also determined to ensure that the test strip has worked properly.

A typical procedure for using the test kit as shown in FIG. 3c comprises: removing the reaction device from a tube; adding 300 microliters (0.3 ml) urine (16) to the tube; replacing the reaction device; ensuring that the conjugate pad is at the base and that the absorbent pad is at the top; allowing the urine (16) to migrate through the device; waiting 5-10 minutes; and reading the result—the assay is complete where there is clear banding without background coloration on the strip. Clear banding means discernable band formation as substantially shown and described in FIGS. 4a, 4b, 4c, and 4d.

Referring now to FIGS. 4a, 4b, 4c, and 4d a schematic diagram is shown illustrating how the results for the competitive assay should be interpreted. FIG. 4a shows the absence of a band in the first reaction site (7) indicating a positive sample. FIG. 4a also shows control reaction site (9), which should always have a line in the completed assay—if no control line is present at reaction site (9) the test should be repeated. FIG. 4b shows clear line formation in first reaction site (7) and a clear line formation in control reaction site (9) indicating respectively a negative result for the analyte of interest, and that the test has worked. FIG. 4c shows a clear line formation at control reaction site (9) and a faint line formation at reaction site (7) illustrating a low positive sample, and that the test has worked. FIG. 4d shows a faint band formation at control reaction site (7) indicating a sample positive for analyte. FIG. 4d also shows clear band formation at the control reaction site indicting that the test has worked. In samples comprising very low BJP titers a faint (ghost) line may be observed. Under these circumstances the sample should be concentrated prior to immunofixation.

The test may be used as a general screen of all potential myeloma samples, or as a more general screen for all senior population and renal referral urines. It may also be used after electrophoresis, where the presence of a band in the gamma/beta region is not easily discernible. The test should not be used for samples with high total protein (>1 g/L), and all samples in this range should be evaluated by electrophoresis. Following a positive result the sample should be further investigated for full characterisation by immunoelectrophoresis or immunofixation electrophoresis.

### EXAMPLE 1

Inoculated Goat Serum is Affinity purified against human BJP extract, suspended in a buffered saline solution and conjugated to 40 nm Gold colloid particles (Veda Labs, Alenco—France) by the following protocol. A ˜40 mn colloidal gold sol solution (externally Quality controlled by EM) is adjusted to pH 6 using 0.2M Sodium Hydroxide solution (using a Beckmann pH meter). The antibodies are introduced in the sol at circa 5-micrograms/ml concentration and incubated for 10 minutes. 10% Bovine serum albumin (Bayer fraction V) in tris buffered saline is then added to a final concentration of 0.2%. The solution is centrifuged for 30 minutes at 4° C. The supernatant is discarded and the pellet resuspended in BSA in Tris buffered saline. The suspension is then introduced into a glycerol gradient column and centrifuged for 45 minutes at 4° C. The purified band is then extracted from the gradient column.

### EXAMPLE 2

Competitive Immunoassay Dipstick

Goat antibodies to the Free and Bound light chain classes were conjugated to 40 nm colloidal gold particles and introduced to a conjugate pad of glass paper. A 6 mm wide strip of cellulose nitrate was striped with individual lines of Protein A and urinary derived monoclonal light chain. An absorbent pad was attached to one end of the cellulose nitrate membrane. The glass pad was compressed against the cellulose nitrate membrane and urine seeded with monoclonal urinary derived light chains at concentrations 0 m/L, 10 mg/L, 20 mg/L, 30 mg/L, 50 mg/L, 100 m/L, 500 mg/L and 1000 mg/L. Urines were also seeded with albumin, IgA, IgG and IgM (polyclonal) at concentrations 0 mg/L, 10 mg/L, 20 mg/L, 30 mg/L, 50 mg/L, 100 m/L, 500 mg/L and 1000 mg/L. 300 microliters of seeded urine was introduced to the glass pad.

Results are classified according to the absence of a clear line (positive) on the cellulose acetate where the competitive light chains were striped. Where the result was unclear an assignation ‘G’ is recorded. In all cases a line was observed at the Protein A line indicating that the conjugate/conjugate-antigen complex had correctly migrated through the test zone.

| | Class | | | | | |
|---|---|---|---|---|---|---|
| mg/l | Kappa | Lambda | IgA | IgG | IgM | Albumin |
| 0 | − | − | − | − | − | − |
| 10 | + | − | − | − | − | − |
| 20 | + | G | − | − | − | − |
| 30 | + | + | G | − | G | − |
| 50 | + | + | + | G | + | − |
| 100 | + | + | + | + | + | − |
| 500 | + | + | + | + | + | − |
| 1000 | + | + | + | + | + | − |

### EXAMPLE 3

### Summary of Clinical Results Test Sensitivity ˜10 mg/L Urinary Free Light Chain

| Sensitivity | Specificity |
|---|---|
| 88%* | 100% |

Assay shows no cross reactivity between light chains, with pH, Sg, Albumin or any other physiological factors. *Samples demonstrated to have low levels of polyclonal (para) protein.

### EXAMPLE 4

Basic Manufacturing Procedure: Inoculated Goat Serum is Affinity purified against human BJP extract, suspended in a buffered saline solution and conjugated to 40 nm Gold colloid particles. Conjugate pad material is liberally soaked in Conjugate solution and cut to 7 mm length then affixed to CN membrane. Absorbent pads are attached to top and base of the device to allow sample introduction and osmotic conductance. Purified Human Bence-Jones Protein is suspended in Buffered saline and applied in a 1 mm line on the CN strip (test zone). This Antiserum is a Goat Polyclonal Anti-Human Free and Bound Light chain (But monoclonal or different host animal would be equally adequate). A 1 mm strip of Buffered saline suspended Protein A is applied at the control line. Strips are cut to a 6 mm width and placed in the sample reaction tube.

What is claimed is:

1. A method for determining the presence of an analyte in urine sample, comprising the steps of:
providing a conjugate pad comprising a chromogenic mobile specific binding partner for an analyte;
providing a chromatographic test strip comprising a matrix through which a urine test sample can flow by capillarity wherein said chromatographic test strip comprises at lease two reaction sites;
a first reaction site comprising a first immobilized specific binding reagent capable of immobilizing said chromogenic mobile specific binding partner in relation to the presence of the analyte in the urine sample; and
a control reaction site comprising a specific binding reagent capable of immobilizing said chromogenic mobile specific binding partner;
contacting said conjugate pad to said chromatographic test strip such that said first reaction site lies between said conjugate pad and said control reaction site;
contacting said chromatographic test strip with an absorbent pad such that said absorbent pad is positioned opposite said conjugate pad and such that both said first reaction site and control reaction site lie in-between said conjugate pad and said absorbent pad;
developing said chromatographic test strip by applying urine sample suspected of containing said analyte thereto thereby allowing the same to contact said chromogenic mobile specific binding partner to form an analyte/chromogenic mobile specific binding partner complex whereby capillarity carries the urine test sample along the strip to the first reaction site containing said immobilized specific binding reagent and said control reaction site comprising said specific binding partner;
determining the presence of analyte in the urine test sample by detecting the presence of chromogenic complex at said first reaction site;
determining if migration has occurred by detecting the presence of chromogenic complex at said control reaction site; wherein detection may be made by observation of color at the control reaction site.

2. The method of claim 1 further comprising a second reaction site positioned in-between said first reaction site and said control reaction site capable of immobilizing said chromogenic mobile specific binding partner in relation to the presence of whole antibody in said urine.

3. The method of claim 1 wherein said analyte is selected form the group consisting of free kappa chains, free and bound kappa, free lambda, and free and bound lambda.

4. The method of claim 1 wherein said urine sample is untreated urine.

5. The method of claim 1 wherein said mobile specific binding partner is at least one conjugated monoclonal antibody.

6. The method of claim 1 wherein said chromogenic mobile specific binding partner is selected from the group consisting of conjugated anti-free and bound kappa antibody and conjugated anti-free and bound lambda antibody.

7. The method of claim 1 wherein said immobilized specific binding reagent is selected from the group consisting of free kappa, free and bound kappa, free lambda, free and bound lambda for performing a competitive analysis.

8. The method of claim 1 wherein said immobilized specific binding reagent is selected from the group consisting of anti-free kappa antibody and anti-free lambda antibody.

9. The method of claim 1 wherein said specific binding reagent is Protein A for the detection of immunochemicals.

10. The method of claim 1 wherein said second reaction site comprises a second specific binding reagent selected from the group consisting of anti-free and bound kappa antibody and anti-free and bound lambda antibody for the determination of the presence of whole antibody.

11. The method of claim 1 wherein said assay is a sandwich assay and the step of determining the presence of analyte in urine further comprises visualization of a band at said first and second reaction site.

12. The method of claim 1 wherein the step of determining the presence of analyte in urine further comprises visualization of said first and said control reaction site, wherein the absence of band formation at said first reaction site indicates a positive result and the visualization of a band at said first reaction site indicates a negative result and wherein the visualization of band formation at said control reaction site indicates that the test has worked in competitive assay.

13. A device for the detection of analyte in urine comprising:
a conjugate pad said conjugate pad comprising a chromogenic mobile specific binding partner capable of binding to an analyte;
a chromatographic test strip comprising a matrix through which urine can pass by capillarity carrying said mobile specific binding partner and said analyte, wherein said chromatographic test strip comprises three reaction sites,
a first reaction site comprising an immobilized specific binding reagent capable of immobilizing said chromogenic mobile specific binding partner in relation to the presence of the analyte in the urine sample,
a second reaction site comprising a second immobilizing specific binding reagent capable of immobilizing said chromogenic mobile specific binding partner in relation to the presence of the analyte in the urine sample,
a third control reaction site comprising a third immobilizing specific binding partner capable of immobilizing said mobile specific binding partner in relation to the capillary action transporting said chromogenic mobile specific binding partner through said chromatographic test strip;
an absorbent pad disposed upon said chromatographic test strip such that said absorbent pad is positioned opposite said conjugate pad and such that said first reaction site, second reaction site, and said third reaction site lie in-between said conjugate pad and said absorbent pad.

14. The device of claim 13 wherein said urine is untreated human urine.

15. The device of claim 13 wherein said analyte is selected form the group consisting of free kappa chains, free and bound kappa, free lambda, and free and bound lambda.

16. The device of claim 13 wherein said chromatographic test strip is a porous material.

17. The device of claim 13 wherein said chromatographic test strip is nitrocellulose or nylon.

18. The device of claim 13 wherein said chromogenic mobile specific binding partner is at least one conjugated monoclonal antibody.

19. The device of claim 13 wherein said chromogenic mobile specific binding partner is a conjugated monoclonal antibody cocktail.

20. The device of claim 13 wherein said chromogenic mobile specific antibody is selected from the group consisting of conjugated anti-free and bound kappa antibody and conjugated anti-free and bound lambda antibody.

21. The device of claim 13 wherein said immobilized specific binding reagent is anti-free kappa antibody.

22. The device of claim 13 wherein said third immobilizing specific binding partner is Protein A.

23. The device of claim 13 wherein said second immobilizing specific binding reagent is selected from the group consisting of anti-free and bound kappa antibody and anti-free and bound lambda antibody for the determination of the presence of whole antibody.

24. The device of claim 13 wherein the presence of analyte in urine is determined by visualization of a band at said first and second reaction site.

25. The device of claim 13 wherein the visualization of the control reaction site indicates that immunological chemicals have migrated through said chromatographic test strip.

26. A test strip for the determination of an analyte in urine comprising:
a backing member;
a chromatographic test strip disposed upon said backing member said chromatographic test strip comprising a matrix through which a urine sample can flow by capillarity wherein said chromatographic test strip comprises at lease two reaction sites,
a first reaction site comprising an immobilized specific binding reagent capable of immobilizing a chromogenic mobile specific binding partner in relation to the presence of the analyte in the urine sample; and
a second reaction site comprising a specific binding reagent capable of immobilizing said chromogenic mobile specific binding partner.

27. The test strip of claim 26 wherein said urine is untreated human urine.

28. The test strip of claim 26 wherein said chromogenic mobile specific binding partner is selected from the group consisting of free kappa, free and bound kappa, free lambda, free and bound lambda.

29. The test strip of claim 26 wherein said first immobilized specific binding reagent is selected from the group consisting of free kappa, free and bound kappa, free lambda, free and bound lambda.

30. The test strip of claim 26 wherein said specific-binding reagent is Protein A.

31. The test strip of claim 26 wherein said analyte is selected from the group consisting of free light chains and classes thereof.

32. The test strip of claim 26 wherein the presence of analyte in urine is determined by the absence of band formation at the first reaction site.

33. A kit for determining the presence of an analyte in urine comprising;
the test strip of claim 26; and
a reaction tube for mixing said test strip with an aliquot of urine.

34. The kit of claim 33 wherein said reaction tube delivers between 100 microliters of urine to 1.0 preferably 300 microliters of urine to said test strip.

35. The kit of claim 33 wherein said reaction further comprising a cap for facilitating disposal of biological waste.

36. A kit for determining the presence of an analyte in urine comprising;
the device of claim 13; and
a reaction tube for contacting aliquots of urine to said device.

37. The kit of claim 36 wherein said aliquot of urine is an amount within the range of 100 microliters of urine to 1.0 ml preferably 300 microliters of urine.

38. The kit of claim 36 wherein said reaction further comprising a cap for facilitating disposal of biological waste.
